(19) United States
(12) Patent Application Publication
(10) Pub. No.: US 2012/0253264 A1
(43) Pub. Date: Oct. 4, 2012

(54) PHOTOCHEMICAL INTERNALIZATION METHOD

(76) Inventors: Jo Klaveness, Oslo (NO); Anders Hogset, Lysaker (NO)

(21) Appl. No.: 13/390,360

(22) PCT Filed: Aug. 16, 2010
(86) PCT No.: PCT/GB10/01548

§ 371 Date: Jun. 20, 2012

(30) Foreign Application Priority Data
Aug. 14, 2009 (GB) 0914286.0

(51) Int. Cl. A61M 37/00

(52) U.S. Cl. 604/20

(57) ABSTRACT

The invention relates to methods of introducing drugs into cells which are located in body cavities. In particular, it provides a photosensitizing agent for use in a method of introducing a drug molecule into the cytosol of a cell located within a body cavity, said method comprising the step of contacting said cell with said photosensitizing agent and said drug molecule, and irradiating the cell with light of a wavelength effective to activate the photosensitizing agent. Such methods are particularly suitable for use in the delivery of cytotoxic drugs in the treatment of cancer, especially bladder cancer, ovarian cancer, cervical cancer, lung cancer, brain cancer, colorectal cancer and cancers of the oral and nasal cavity.

The present invention relates to methods of introducing drugs into cells which are located in body cavities. More particularly, it relates to such methods which involve the use of a photosensitizing agent and irradiation of the cells with light of a wavelength effective to activate the photosensitizing agent.

Photochemotherapy or photodynamic therapy (PDT) is a technique for the treatment of various abnormalities or disorders. PDT can be used for treatment of disorders of the skin or other epithelial organs or mucosa, especially cancer or pre-cancerous lesions. It also finds use in the treatment of non-cancerous conditions, such as acne and age-related macular degeneration. PDT involves the application of a photosensitizing agent to the affected area of the body, followed by exposure of the area to photoactivating light in order to activate the photosensitizing agent. Activation of the photosensitizing agent converts this into a cytotoxic form which kills or otherwise reduces the proliferative potential of the affected cells.

A range of photosensitizing agents are known for use in PDT. Those known for clinical use include 5-aminolevulinic acid (5-ALA), 5-ALA methyl ester, 5-ALA hexyl ester, verteporfin, psoralens and porfimer. 5-ALA (Levulan®) and 5-ALA methyl ester (Metvix®) are used for treatment of various dermal conditions; 5-ALA hexyl ester (Hexvix®) is used for diagnosis of urinary bladder cancer; verteporfin (Visudyne®) is used for treatment of macular degeneration in the eye; and porfimer (Photofrin®) is used for treatment of lung cancer and palliative treatment of obstructive oesophageal cancer.

Photochemical internalization (also known simply as “PCI”) is a drug delivery method which involves the use of light and a photosensitizing agent for introducing otherwise membrane-impermeable drugs into the cytosol of a cell, but which does not necessarily result in cell destruction or cell death. In this method, the molecule to be internalized or transferred is applied to the cells in combination with a photosensitizing agent. Exposure of the cells to light of a suitable wavelength activates the photosensitizing agent which in turn leads to disruption of the intracellular compartment membranes and the subsequent release of the molecule into the cytosol. In PDT it is the effect of the light on the photosensitizing agent which forms cell-toxic materials that directly affect the disease. In contrast, in PCI, the interaction between the photosensitizing agent and light is used to affect the cell such that intracellular uptake of the drug is improved. Both mechanisms go through a pathway involving singlet oxygen species. Singlet oxygen is a highly reactive form of oxygen that can oxidize various biomolecules, including molecules in the cellular membranes. In PDT a direct-acting therapeutic agent is not normally used, while in PCI a direct-acting drug (or prodrug thereof) is always used in conjunction with the photosensitizing agent. Drugs which may be considered to be “direct-acting” are those which have an inherent biological activity (whether therapeutic or prophylactic). When present in vivo at the desired target site, such drugs do not require light to be active. The photosensitizing agents which may be used in PCI might also be used in PDT, however, not all PDT-active photosensitizers can be used in PCI.

PCI is described in the following patent documents: WO 96/07432, WO 00/54708, WO 02/44396, WO 02/44395, WO 03/020309, U.S. Pat. No. 6,680,301 and U.S. Pat. No. 5,876,989. The technology is further described in the following publications: Berg, K. et al. in Cancer Res. (1999) 59, 1180-1183, Høgset, A. et al. in Hum. Gene Ther. (2000) 11, 869-880, Prasmickaite, L. et al. in J. Gene Med. (2000) 2, 477-488, Selbo, P. K. et al. in Biochim. Biophys. Acta (2000) 1475, 307-313, Selbo, P. K. et al. in Int. J. Cancer (2000) 87, 853-859, Selbo, P. K. et al. in Int. J. Cancer (2001) 92, 761-766, Berg, K. et al. in Photodynamics News (2001) 4, 2-5, Prasmickaite, L. et al in Photochem. Photobiol. (2001) 73, 388-395, Selbo, P. K. et al. in Photochem. Photobiol. (2001) 74, 303-310, Selbo. P. K. et al in Tumor Biol. (2002) 23, 103-112, Høgset, A. et al. in Adv. Drug Deliv. Rev. (2004) 56, 95-115, Berg, K et al. in Curr. Opin. Mol. Ther. (2004) 6, 279-287, Prasmickaite, L. et al. in Expert Opin. Mol. Ther. (2004) 4, 1403-1412, Berg, K. et al. in Clin. Cancer. Res. (2005) 11, 8476-8485, Berg, K. et al. in Curr. Pharmacol. Biotech (2006) 8, 362-372 and Weyergang, A. et al. in Photochem. Photobiol. Sci. (2008) 7, 1032-1040.

The ability to deliver drugs directly to the site of intended action is important in any method of medical treatment. Over the years, a variety of devices and methods have been developed to deliver drugs in a more targeted manner ensuring that the desired level of drug reaches the desired site. Various publications describe drug delivery directly into body cavities, including U.S. Pat. No. 5,292,516 and U.S. Pat. No. 6,346,272 which describe body cavity drug delivery using thermoreversible gels and thermo-irreversible gels, respectively. WO 2007/021964 describes an intravesical (into the urinary bladder) drug delivery device and method, and EP-A-0937478 describes a device and apparatus for intracavity drug delivery during video-assisted surgery or other endoscopic procedures. There are, however, relatively few products in clinical use for drug delivery into body cavities.

Generally, the most popular route of administration of drugs is oral where the drug is provided in the form of tablets or capsules. Most oral drug formulations are intended for systemic uptake of the drug substance. Another enteral administration form is rectal administration in which the drug may be administered in the form of suppositories or an enema.

Certain drugs may be administered topically directly onto the skin in the form of a cream or lotion. In contrast to oral medicaments, these are usually intended for treatment of a local disease, such as acne or other skin diseases. Drugs for the treatment of diseases in body cavities include those known for treating lung diseases, such as asthma, which are frequently administered by inhalation. Antibiotics for the treatment of ear and eye infections can be administered locally by application of ear and eye drops. Other common routes of administration of drugs are injections or infusions, such as intravascular injections and infusions (into veins), intramuscular injections (into muscles) and subcutaneous injections (under the skin)

Each route of administration has advantages and disadvantages. The main advantage of oral administration of systemic drugs is patient compliance (user friendliness), whereas the main advantage associated with intravascular administration relates to its safe pharmacokinetics. Both the pharmacodynamic and pharmacokinetic properties (i.e. clinical efficacy) and the toxicological profile of drugs are generally very dependent on the route of administration.

Administration of drugs directly into body cavities (“intracavitary” drug delivery), such as for example intravitreal (injection into the eye), intranasal (into the nose), intravaginal (into the vagina), intraperitoneal (injection into the peritoneum), and intravesicular (into the urinary bladder), is uncommon. A particular problem with the administration of drugs into body cavities relates to the pharmacokinetic properties, in particular cellular uptake and drug absorption.

The present inventors have now found that photochemical internalization (PCI) is particularly effective for use in the delivery of drugs into cells and tissues located in body cavities. Surprisingly, they have found that cellular uptake and absorption of drugs in body cavities can b greatly improved, when compared to conventional drug delivery methods, by using a photosensitizing agent in combination with irradiation with light. As a result, such methods are associated with improved therapeutic efficacy and improved safety.

Viewed from one aspect the invention thus provides a method for introducing a drug molecule into the cytosol of a cell located within a body cavity, said method comprising contacting said cell with said drug molecule and a photosensitizing agent, and irradiating the cell with light of a wavelength effective to activate the photosensitizing agent.

In a further aspect the invention provides a photosensitizing agent for use in a method of introducing a drug molecule into the cytosol of a cell located within a body cavity, wherein said method comprises the step of contacting said cell with said photosensitizing agent and said drug molecule, and irradiating the cell with light of a wavelength effective to activate the photosensitizing agent.

The methods and compositions herein described may be used to improve the uptake of any drug molecule in any body cavity of the human or non-human, preferably mammalian, body. Preferred drugs are, however, those which do not readily penetrate cell membranes in the absence of light. As used herein, the term “body cavity” is considered to encompass not only natural body cavities, but also those which may be artificially created, e.g. by injury or by means of a surgical procedure.

The invention may, for example, be used within body cavities such as the urinary bladder (e.g. the urethra), the oral cavity, the nasal cavity, in the female reproductive body cavity system (e.g. the vaginal cavity), in the abdominal cavity (peritoneum), in the lower part of the gastrointestinal system (e.g. in the rectum and/or colon), and in the cranial cavity. Other body cavities in which the invention finds use include the eye (intravitreous), the lungs and bronchial system and cavities generated after surgery (e.g. following tumor surgery).

The photosensitizing agent to be used according to the invention may be any known photosensitizing agent which localises to intracellular compartments, particularly endosomes or lysosomes. A range of suitable agents are known in the art and described in the literature for use in PCI, including in WO 96/07432, WO 03/020309 and in GB-A-2420784. These include, in particular, phthalocyanines such as di-sulphonated aluminium phthalocyanines (e.g. AlPcS₂ and AlPcS₂ₐ); sulphonated tetraphenylporphyrins (e.g. TPPS₂ₐ, TPPS₄, TPPS₁ and TPPS₂ₒ); nile blue; chlorins and chlorin derivatives including bacteriochlorins and ketochlorins; uroporphyrin I; phylloerythrin; natural and synthetic porpyhrins including hematoporphyrin and benzoporphyrins; methylene blue; cationic dyes; tetracyclines, naphthalocyanines; texaphyrines; pheophorbides; purpurins; rhodamines; fluoresceins; lysosomotropic weak bases; and porphycenes.

More preferred for use in the invention are photosensitizers with amphiphilic properties, cationic photosensitizers and anionic photosensitizers. The following are among the most preferred photosensitizers for use in the invention: TPCS₂ₐ, TPPS₂ₐ, AlPcS₂ₐ, TPPS₄ and porfimer (Photofrin®).

Photosensitizers suitable for use in the invention include pharmaceutically acceptable salts of any of the photosensitizing agents herein described. Particularly preferred are salts of amphiphilic photosensitizers which have enhanced water solubility (preferably a water solubility of at least 0.5 mg/ml); these are particularly suitable for parenteral administration, for example in the form of aqueous solutions. Such salts are described in the applicant's co-pending UK patent application No. 0914287.8, the entire contents of which are incorporated herein by reference. Salts having a solubility in water which exceeds 1 mg/ml, preferably 3 mg/ml, more preferably 5 mg/ml, e.g. 10 mg/ml, are particularly preferred for use in the methods herein described. Salts for use in the invention may have a solubility of at least 20 mg/ml, more preferably at least 25 mg/ml, e.g. at least 30 mg/ml.

Preferred salts for use in the invention may be formed from a pharmaceutically acceptable base such as an organic amine, in particular an amino alcohol (or alkanolamine). As used herein, the term “amino alcohol” is intended to include any organic compound containing both at least one amine functional group and at least one alcohol functional group. The amino alcohols may be linear, branched or cyclic. Among the amino alcohols which are particularly suitable for the preparation of the salts for use in the methods herein described are the lower aliphatic amino alcohols such as monoethanolamine, di-ethanolamine, tri-ethanolamine and 2-amino-2-(hydroxymethyl) propane-1,3-diol, etc. Other suitable amino alcohols include cyclic compounds such as 4-(2-hydroxyethyl)-morpholine and 1-(2-hydroxyethyl)-pyrrolidine. Particularly preferred for use in the invention are the basic salts with the amino sugars glucamine and N-methylglucamine (meglumine). Particularly preferred salts for use in the invention are the N-methylglucamine salts and ethanolamine salts.

Alternatively, the salt for use in the invention may be a pharmaceutically acceptable acid addition salt. Suitable salt forming acids are sulphonic acids and derivatives of such acids which are capable of forming salts with cationic photosensitizers.

As used herein, the term “sulphonic acid” is intended to include any organic compound containing at least one —SO₃H group. This may comprise 1, 2 or 3-SO₃H groups, most preferably 1 or 2, e.g. 1. The term “derivatives”, when used in relation to sulphonic acid is intended to encompass any such compounds containing at least one (preferably 1, 2 or 3, most preferably 1 or 2, e.g. 1) —SO₃X group (where X is a physiologically tolerable cation, such as a sodium, calcium, potassium, magnesium or meglumine cation).

Acid addition salts for use according to the invention will typically be derived from a cationic photosensitizing agent and a mono-protic sulphonic acid such as methane sulphonic acid, thereby forming a 1:1 salt. Alternatively, salts may be formed between the photosensitizer and a di- or tri-protic sulphonic acid, such as ethane-1,2-disulfonic acid. In the case where an acid having more than one acidic proton is used, the resulting salt may have a stoichiometric ratio other than 1:1, for example 2:1 (photosensitizer:acid) or 3:1 (photosensitizer:acid).

Sulphonic acids and sulphonic acid derivatives suitable for use in forming the salts for use according to the invention include those of formulae R—SO₃H (I) and R—SO₃X (II) in which R may be a hydrogen atom or an optionally substituted alkyl (e.g. a C₁₋₂₀ alkyl group) or aryl group (e.g. an aryl group of up to 20 carbon atoms), preferably an optionally substituted alkyl or aryl group.

As used herein, the term “alkyl” includes any long or short chain, straight-chained, branched or cyclic aliphatic, saturated or unsaturated hydrocarbon group. Optionally, this group may be substituted (e.g. mono- or poly-substituted), for example by hydroxy, alkoxy, acyloxy, nitro, alkoxycarbonyloxy, amino, aryl, oxo or halo (e.g. fluoro or chloro) groups. The unsaturated alkyl groups may be mono- or polyunsaturated and include both alkenyl and alkynyl groups. Preferred salts for use in accordance with the invention are those formed from acids of formulae (I) or (II) in which R is an optionally substituted (i.e. mono- or poly-substituted), linear, branched or cyclic (e.g. mono- or bicyclic, bridged or non-bridged) alkyl group which may contain up to 20 carbon atoms, or an optionally substituted (i.e. mono- or poly-substituted) aryl group, which preferably contains up to 20 carbon atoms. Preferred substituents which may be present in group R include C₁₋₆ alkyl (e.g. methyl), hydroxy, alkoxy, acyloxy, nitro, alkoxycarbonyloxy, amino, aryl, oxo and halo (e.g. fluoro or chloro).

In general, salts for use according to the invention that are formed between a photosensitizing agent and a sulphonic acid compound comprise a single sulphonic acid moiety, i.e. a mono-protic acid. However, as noted above, salts formed from acids having more than one sulphonic acid moiety (e.g. 2 or 3 such groups) may also be used. Other substituents which may be present in group R therefore include one or more, preferably one, —SO₂OH, —SO₂OX (where X is as hereinbefore defined) or —SO₂O⁻ group. Representative examples of disulphonic acids which may be used to prepare the salts for use according to the invention include ethane-1,2-disulphonic acid and napthalene-1,5-disulphonic acid.

Preferred alkyl groups for group R may contain up to 20, but preferably up to 15, e.g. up to 12 carbon atoms. However, alkyl groups containing up to 10, e.g. up to 5, more preferably 1, 2 or 3 carbon atoms are preferred. In particular, linear alkyl groups having up to 10 carbon atoms are preferred, e.g. methyl, ethyl or propyl groups. Although these groups may be substituted or unsubstituted, preferably these will be unsubstituted.

Preferred aryl groups for group R include optionally substituted phenyl or napthyl groups. Preferably the aryl group is substituted, for example by one or more (e.g. by one, two or three) substituents which may include C₁₋₆ alkyl groups (preferably C₁₋₄ alkyl, e.g. methyl), alkoxy (e.g. methoxy), nitro, halo (e.g. fluoro or chloro), —SO₃H, —SO₃X (where X is as hereinbefore defined), —SO₂O⁻ or trifluoromethyl groups. Representative examples of aryl groups include toluene (e.g. p-toluene), benzene, napthalene and napthalene sulphonate (e.g. 2-napthalene sulphonate).

Examples of sulphonic acids suitable for forming the acids for use in the present invention include: ethane-1,2-disulphonic acid, ethanesulphonic acid, 2-hydroxy-ethanesulphonic acid, methanesulphonic acid and naphthalene-1,5-disulphonic acid.

Examples of preferred salts for use in the invention for PCI delivery of drugs to body cavities include the following:
TPCS₂ₐ diethanolamine salt
TPCS₂ₐ ethanolamine salt
TPCS₂ₐ N-methyl-glutamine salt
TPCS₂ₐ triethanolamine salt
TPCS₂ₐ 1-(2-hydroxymethyl)-pyrrolidine salt
TPCS₂ₐ 2-amino-2-(hydroxymethyl) propane-1,3-diol salt
TPPS₂ₐ diethanolamine salt
TPPS₂ₐ ethanolamine salt
TPPS₂ₐ N-methyl-glucamine salt
TPPS₂ₐ triethanolamine salt
TPPS₂ₐ 1-(2-hydroxymethyl)-pyrrolidine salt
TPPS₂ₐ 2-amino-2-(hydroxymethyl)propane-1,3-diol salt
Porfimer diethanolamine salt
Porfimer ethanolamine salt
Porfimer N-methyl-glucamine salt
Porfimer triethanolamine salt
Porfimer 1-(2-hydroxymethyl)-pyrrolidine salt
Porfimer 2-amino-2-(hydroxymethyl)propane-1,3-diol salt.

The photosensitizing agent will generally be provided for use in the form of a pharmaceutical composition. Such compositions comprise a photosensitizing agent as herein described, in combination with at least one pharmaceutical carrier or excipient, and may either be solid, liquid or semi-liquid formulations. Solid formulations include powders (e.g. for inhalation or for incorporation into a capsule for oral administration), tablets, etc. Semi-liquid formulations include gels, creams and lotions. Liquid formulations include solutions, suspensions, droplets (e.g. for inhalation), emulsions, etc.

Additional components for use in the compositions include any pharmaceutically acceptable additive. Typically, these may be solvents such as water, alcohols, glycerol, polyethyleneglycols, various salts and other pharmaceutically acceptable salts, agents for adjusting osmotic pressure, buffers, e.g. phosphate salts or tris, stabilizing compounds such as antioxidants, or compounds with an effect on the viscosity of the composition, e.g. polysaccharide derivatives. Mucoadhesive agents may also be used in the compositions particularly where these are to be directly administered to a mucosa-lined body cavity. Mucoadhesive agents are well known and described in the literature and any of these may be used in the invention. Preferred mucoadhesives include polyacrylic hydrogels, chitosan, polyvinyl alcohol, hydroxypropyl cellulose, hydroxypropyl methyl cellulose, sodium alginate, scleroglucan, xanthan gum, pectin, orabase and polygalactonic acid.

The compositions for use according to the invention can be sterile or non-sterile. However, for use in all body cavities other than the gastrointestinal system (which includes the oral cavity), the compositions should be sterile. Methods of sterilization include autoclaving, dry head sterilization, gamma-sterilization and treatment with ethylene oxide.

The compositions herein described may be provided in “ready-to-use” form, for example in which the photosensitizer is already dissolved in a suitable solvent such as an aqueous solution. Alternatively, this may be provided in dry (e.g. powdered) form with instructions for dissolving this in an aqueous solution with stirring prior to use.

For use in PCI, the compositions herein described will be administered in combination with a therapeutic agent (also herein referred to as “drug molecules”). Depending on the condition to the treated, the nature of the composition, etc., the photosensitizing agents may be co-administered with the drug molecules, for example in a single composition, or they may be administered sequentially or separately.

Viewed from a further aspect the invention thus provides a product comprising a photosensitizing agent as herein described, together with a therapeutic agent for simultaneous, separate or sequential use in a method of photochemical internalization in which the therapeutic agent is introduced into the cytosol of a cell located within a body cavity.

Alternatively viewed, this aspect of the invention also provides a kit for use in a method of photochemical internalization in which the therapeutic agent is introduced into the cytosol of a cell located within a body cavity, said kit comprising:
(a) a first container containing a photosensitizing agent as herein described; and
(b) a second container containing a therapeutic agent.

In the case where the photosensitizing agent is intended for use as a solution, e.g. as an aqueous solution, the kit may contain one or both of the photosensitizer and the drug in dry form, together with a further container (third container) containing an aqueous solution. The photosensitizer and/or the drug may then be dissolved or suspended in the aqueous solution prior to use. Preferably, the photosensitizer will be substantially dissolved in the solution at the time of administration, especially where this is to be administered parenterally.

The terms “photochemical internalization” and “PCI” are used herein to refer to the cytosolic delivery of molecules (e.g. drug molecules) which includes the step of release of molecules from intracellular/membrane bound compartments into the cytosol of the cells of a patient.

The drug molecule to be translocated into intracellular compartments of the cells of the patient and the photosensitizing agent may be applied to the cells located within the desired body cavity together or sequentially, upon which the photosensitizing compound and the molecule are endocytosed or in other ways translocated into endosomes, lysosomes or other intracellular membrane restricted compartments. The molecule to be internalized within the cells located in the desired body cavity is released by exposure of the cells to light of suitable wavelengths to activate the photosensitizing compound which in turn leads to the disruption of the intracellular compartment membranes and the subsequent release of the molecule into the cytosol.

The precise timing of the addition of the molecule to be transferred (i.e. the drug molecule) and photosensitizing agent and timing of irradiation to achieve the above described effects needs to take into account various factors including the cells to be treated, the nature of the drug molecules, the environment of the cells, and whether administration is direct to the target tissue or at a distal site. Taking these considerations into account appropriate timings may readily be determined by those skilled in the art. Typically, the drug molecule and the photosensitizing agent will be contacted with the cells prior to irradiation. Light irradiation may be effected any time after administration of the photosensitizing agent. In general, the drug molecule and photosensitizing agent may be applied either simultaneously or separately from 1 to 72 hours prior to irradiation, preferably 4 to 48, e.g. 4 to 24 hours prior to irradiation.

However, irradiation may be performed before the drug molecule has been taken up into the same intracellular compartment of the cell as the photosensitizing agent (see WO 02/44396 which describes how this may be achieved in more detail), e.g. by irradiation before administration of the drug molecule, e.g. by adding the drug molecule 5 minutes to 24 hours, for example, 30 minutes to 2 hours, after irradiation.

In certain cases, the drug molecule will be administered simultaneously with the photosensitizing agent. This may be achieved by administration to the patient of a pharmaceutical composition which comprises a photosensitizing agent as herein described, together with a therapeutic agent. In such a composition, a pharmaceutically acceptable carrier or excipient may additionally be present.

Alternatively, and more typically, the photosensitizer may be administered prior to administration of the drug molecules.

In a yet further aspect the invention provides a pharmaceutical composition comprising a photosensitizing agent as herein described, together with a therapeutic agent, for use in a method of treating cells located within a body cavity, e.g. a method of treating cancer or a method of gene or oligonucleotide (e.g. siRNA) therapy, in which said composition is contacted with cells or tissues of a patient located within said body cavity and said cells or tissues are irradiated with light of a wavelength effective to activate said photosensitizing agent.

In a still yet further aspect the invention provides the use of a photosensitizing agent as herein described and/or a therapeutic agent for the preparation of a medicament for use in a method of treating cells located within a body cavity, e.g. a method of treating cancer or a method of gene or oligonucleotide (e.g. siRNA) therapy, in which said photosensitizing agent and said therapeutic agent are contacted (either separately, simultaneously or sequentially) with cells or tissues of a patient located within said body cavity and said cells or tissues are irradiated with light of a wavelength effective to activate said photosensitizing agent.

The photosensitizing agents herein described may be used for transporting or transfecting any drug molecule into the cytosol of living cells which are located in a body cavity. These may be used not only to transfer molecules (or parts or fragments thereof) into the interior of a cell but also, in certain circumstances, to present or express them on the cell surface. Thus, following transport and release of a drug molecule into the cell cytosol, if the cell(s) in question are specialised cells, such as for example antigen presenting cells, the molecule or fragment, may be transported to the surface of the cell where it may be presented on the outside of the cell, i.e. on the cell surface. Such methods have particular utility in the field of vaccination, where vaccine components, i.e. antigens or immunogens, may be introduced into a cell for presentation on the surface of that cell, in order to induce, facilitate or augment an immune response. Further details as to the utility of expressing molecules on the cell surface are described in WO 00/54802.

The drug molecules which can be introduced into the cytosol of cells using the photosensitizing agents herein described include molecules which do not readily penetrate cell membranes. Additionally, the agents herein described can increase the cytosol delivery and activity of drug molecules which are only partly able to penetrate the membrane of the cell or the membranes of intracellular vesicles. Drug molecules may be organic compounds, proteins or fragments of proteins such as for example peptides, antibodies or antigens or fragments thereof. Another class of drug molecules which may be introduced using the agents herein described are cytotoxic drugs such as protein toxins or cytotoxic organic compounds. Molecules which may be of clinical interest for treatment of cancer, but are restricted by low or no uptake into the cytosol can be introduced into the cytosol and targeted to specific cells when using the methods herein described. Gelonin is an example of such a molecule. A further example of a cytotoxic agent which may be used in conjunction with the photosensitizing agents herein described is bleomycin.

Many pharmaceutically active drugs may be delivered to the cells located within body cavities using the methods and compositions herein described. Suitable classes of drugs which can be administered include, for example, anti-cancer drugs, antibacterial substances, anti-virals, anti-fungal agents, immune-modulating drugs, anti-inflammatories, analgesics, gene therapy agents, oligonucleotides and other gene expression modifying agents.

In a preferred embodiment of the invention the drug for delivery to the cells within a body cavity may be an anti-cancer drug. This may be a natural product, for example a cytotoxic antibiotic derivative, a semi-synthetic or synthetic product. Suitable anti-cancer drugs include biomolecules prepared by recombinant technology or other biotechnology-based production methods. Anti-cancer drugs which specifically target gene products over-expressed in target cells, which target a cellular protein-based target or a cellular non-protein-based target, e.g. nucleic acids or other non-protein cell components, or which target an enzyme are particularly preferred for use in the invention.

Particular forms of cancer which may be treated in accordance with the methods herein described include bladder cancer, ovarian cancer, cervical cancer, lung cancer, brain cancer, colorectal cancer and cancers of the oral and nasal cavity.

The drug for delivery to the cells within a body cavity may alternatively be a drug for the treatment of infections, for example infections caused by a virus, by bacteria or by fungi. Such drugs are particularly suitable for delivery into the cells located within the nose (intranasal).

Drugs with immune-modulating properties may also be used in the invention. These include protein or peptide antigens for vaccine or immune-stimulating purposes, immune-stimulating oligonucleotides, genes encoding antigens or immune-modulating proteins or peptides, oligonucleotides modifying gene expression in the immune response system, and small molecule drugs having immune-modulating properties like methotrexate, azathioprine, lenalidomide, cyclosporine and mycophenolic acid.

Another class of appropriate drug molecules are nucleic acids. Nucleic acids may be used in the form of genes encoding for example therapeutic proteins, antisense RNA molecules, ribozymes, RNA aptamers, short hairpin RNAs (shRNAs), microRNAs or triplex forming oligonucleotides. Alternatively the nucleic acids may be employed in the form of non-encoding molecules such as for example synthetic DNA or RNA antisense molecules, ribozymes, siRNAs, microRNAs, aptamers, triplex forming oligonucleotides, peptide nucleic acids (PNAs), transcription factor “decoy” DNA or chimeric oligonucleotides for repair of specific mutations in the patient. Where appropriate the nucleic acid molecules may be in the form of whole genes or nucleic acid fragments optionally incorporated into a vector molecule e.g. a plasmid or a viral vector. The latter form has particular applicability when the transfer molecule is to be used in methods of gene therapy in which genes are therapeutically transferred to a patient's cells. This may be used in treating many diseases such as cancer, viral infections, and monogenic disorders such as cystic fibrosis.

Optionally, one or other or both of the photosensitizing agent and the drug molecule to be introduced into the cells may be attached to or associated with or conjugated to carrier molecules, targeting molecules or vectors which can act to facilitate or increase the uptake of the photosensitizing agent or the drug molecule or can act to target or deliver these entities to a particular cell type, tissue or intracellular compartment. Examples of carrier systems include polylysine, chitosans, polyethylenimines or other polycations, dextran sulphate, different cationic lipids, liposomes, reconstituted LDL-particles or sterically stabilised liposomes. These carrier systems can generally improve the pharmacokinetics and increase the cellular uptake of the drug molecule and/or the photosensitizing agent and may also direct the drug molecule and/or the photosensitizing agent to intracellular compartments that are especially beneficial for obtaining photochemical internalization, but they do not generally have the ability to target the drug molecule and/or the photosensitizing agent to specific cells (e.g. cancer cells) or tissues.

However, to achieve such specific or selective targeting the carrier molecules, the drug molecule and/or the photosensitizer may be associated with, bound or conjugated to specific targeting molecules that will promote the specific cellular uptake of the drug molecule into desired cells or tissues. Such targeting molecules may also direct the drug molecule to intracellular compartments that are especially beneficial for obtaining photochemical internalization.

Many different targeting molecules can be employed, e.g. as described in Curiel, D. T. (1999), Ann. New York Acad. Sci. 886, 158-171; Bilbao, G. et al. (1998), in Gene Therapy of Cancer (Walden et al., eds., Plenum Press, New York), Peng K. W. and Russell S. J. (1999), Curr. Opin. Biotechnol. 10, 454-457; and Wickham T. J. (2000), Gene Ther. 7, 110-114.

The carrier molecule and/or the targeting molecule may be associated, bound or conjugated to the drug molecule, to the photosensitizing agent or both, and the same or different carrier or targeting molecules may be used. Such targeting molecules or carriers may also be used to direct the drug molecule to particular intracellular compartments especially beneficial for the employment of PCI, for example lysosomes or endosomes.

The compositions of the invention may be formulated in conventional manner with one or more physiologically acceptable carriers or excipients according to techniques well known in the art. The nature of the composition and carriers or excipient materials, dosages etc. may be selected in routine manner according to choice and the desired route of administration, purpose of treatment, etc. Dosages may likewise be determined in routine manner and may depend upon the nature of the drug molecule, purpose of treatment, age of patient, mode of administration, etc.

Compositions will generally be administered topically or systemically. Topical compositions include gels, creams, ointments, sprays, lotions, pessaries, aerosols, drops, solutions and any of the other conventional pharmaceutical forms in the art. Topical administration to cells or tissues within body cavities which are less readily accessible may be achieved by techniques known in the art, e.g. by the use of catheters or other appropriate drug delivery systems.

Preferably, the compositions may be provided in a form adapted for parenteral administration, for example by intraperitoneal injection, or by infusion. Alternative pharmaceutical forms thus include suspensions and solutions containing the photosensitizing agent optionally together with one or more inert conventional carriers and/or diluents. Formulations for parenteral administration may be in the form of aqueous or non-aqueous, isotonic, sterile injection solutions or suspensions. These solutions may be prepared from sterile powders or granules using one or more carriers or excipients, for example, suitable dispersing, wetting or suspending agents. Suitable carriers for the preparation of solutions for injection include water, saline and dextrose.

Other non-toxic parenterally acceptable diluents or solvents may be used, including amino acid solutions, such as Glavamin® (Fresenius Kabi), carbohydrate solutions such as Glucos® (Braun), electrolytes such as sodium chloride solutions, Ringer's solution, trometamol solutions, or mixtures of any of the foregoing.

The total dose, concentration and administration volume of photosensitizer and drug will vary over a large range depending on several factors. The main factors are: indication (nature of the disease), stage of disease, organ system and choice of photosensitizer and drug.

The concentration of the compounds as described hereinbefore in the compositions depends upon the intended use of the compound, the nature of the composition, mode of administration, the condition to be treated and the patient and may be varied or adjusted according to choice. For use in PCI, it is important that the concentration of the photosensitizing agent is such that once taken up into the cell, e.g. into, or associated with, one or more of its intracellular compartments and activated by irradiation, one or more cell structures are disrupted, e.g. one or more intracellular compartments are lysed or disrupted. The photosensitizing agents may be used at a concentration of, for example, 0.5 to 100 mg per ml. For in vivo human treatments the photosensitizing agent may be used in the range 0.05-20 mg/kg body weight when administered systemically or 0.1-20% in a solvent for topical application. The time of incubation of the cells with the photosensitizing agent (i.e. the “contact” time) can vary from a few minutes to several hours, e.g. even up to 48 hours or longer. The time of incubation should be such that the photosensitizing agent is taken up by the appropriate cells. The incubation of the cells with the photosensitizing agent may optionally be followed by a period of incubation with photosensitizer free medium before the cells are exposed to light and/or the drug molecule is administered.

Determining the appropriate doses of drug molecules for use in accordance with the present invention is routine practice for a person skilled in the art. Where the drug molecule is a protein or peptide, the drug molecules would generally be used at doses of less than 5 mg/kg (e.g. 0.1-5 mg/kg). Where the drug molecule is a nucleic acid, approximately 10⁻⁶-1 g nucleic acid per injection may be used in humans.

Following administration of a compound or composition as herein described the area treated is exposed to light to achieve the desired effect. The light irradiation step to activate the photosensitizing agent may be effected according to techniques and procedures well known in the art. Suitable light sources capable of providing the desired wavelength and light intensity are also well known in the art. The time for which the cells are exposed to light in the methods of the present invention may vary. For example, the efficiency of the internalization of the drug molecule into the cytosol appears to increase with increased exposure to light. Generally, the length of time for the irradiation step is in the order of minutes to several hours, e.g. preferably up to 60 minutes e.g. from 1 to 30 minutes, e.g. from 0.5 to 3 minutes or from 1 to 5 minutes or from 1 to 10 minutes e.g. from 3 to 7 minutes, and preferably approximately 3 minutes, e.g. 2.5 to 3.5 minutes. Appropriate light doses can be selected by a person skilled in the art and will depend on the amount of photosensitizer accumulated in the target cells or tissues. The irradiation will in general be applied at a dose level of 40 to 200 Joules/cm², for example at 100 Joules/cm² at a fluence range of less than 200 mW/cm². Irradiation with wavelengths of light in the range 500-750 nm, e.g. 550 to 700 nm, is particularly suitable for in vivo use in the methods herein described.

Methods for irradiation of different areas of the body, including body cavities, e.g. by lamps or lasers are well known in the art (see for example Van den Bergh, Chemistry in Britain, May 1986p. 430-439). For inaccessible regions this may conveniently be achieved using optical fibres. For some uses, various devices such as catheters may be required for light delivery to areas of interest.

The invention will now be described in more detail by way of the following non-limiting Examples:

### EXAMPLE 1

### Preparation of Meso-Tetraphenyl Porphyrin Disulphonate Bis(Monoethanolamine) ((MEA)₂-TPPS₂ₐ)

Meso-tetraphenyl porphyrin disulphonate bis(triethylamine) prepared from the free acid was dissolved in methanol and an excess of ethanolamine added. The solution was stirred for 15 minutes before the solvent was removed in vacuo at 30° C. with a rotary evaporator. This procedure was repeated two more times.

### EXAMPLE 2

### Preparation of Meso-Tetraphenyl Porphyrin Disulphonate Bis(Meglumate) ((Megl)₂-TPPS₂ₐ)

Meso-tetraphenyl porphyrin disulphonate (200 mg, 0.26 mmol) was added to a solution of N-methyl-D-glucamine (102 mg, 0.52 mmol) in de-ionized water (5 ml) at room temperature. The mixture was stirred for 15 minutes and the mixture was freeze-dried overnight. The title compound was isolated as a dark red solid material. Yield: 310 mg (100%).

### EXAMPLE 3

### Preparation of Meso-Tetraphenyl Porphyrin Disulphonate Bis(Tris(Hydroxymethyl)Methylamine) ((TRIS)₂-TPPS₂ₐ)

Meso-tetraphenyl porphyrin disulphonate (200 mg, 0.26 mmol) was added to a solution of tris(hydroxymethyl)methylamine (63 mg, 0.52 mmol) in de-ionized water (5 ml) at room temperature. The mixture was stirred for 15 minutes and the mixture was freeze-dried overnight.

The title compound was isolated as a dark red solid material. Yield: 260 mg (100%).

### EXAMPLE 4

### Preparation of Meso-Tetraphenyl Porphyrin Disulphonate Bis(Diethanolamine) ((DEA)₂-TPPS₂ₐ)

Meso-tetraphenyl porphyrin disulphonate (100 mg, 0.13 mmol) was added to a solution of diethanolamine (27 mg, 0.26 mmol) in de-ionized water (5 ml) at room temperature. The mixture was stirred for 15 minutes and the mixture was freeze-dried overnight. The title compound was isolated as a dark red solid material. Yield: 103 mg (80%).

### EXAMPLE 5

### Preparation of Meso-Tetraphenyl Porphyrin Disulphonate Bis(1-(2-Hydroxyethyl)Pyrrolidine) ((HEP)₂-TPPS₂ₐ)

Meso-tetraphenyl porphyrin disulphonate (100 mg, 0.13 mmol) was added to a solution of 1-(2-hydroxyethyl)pyrrolidine) (30 mg, 0.26 mmol) in de-ionized water (5 ml) at room temperature. The mixture was stirred for 15 minutes and the mixture was freeze-dried overnight. The title compound was isolated as a dark red solid material. Yield: 117 mg (90%).

### EXAMPLE 6

### Preparation of Meso-Tetraphenyl Porphyrin Disulphonate Bis(Triethanolamine) ((TEA)₂-TPPS₂ₐ)

Meso-tetraphenyl porphyrin disulphonate (100 mg, 0.13 mmol) was added to a solution of triethanolamine (39 mg, 0.26 mmol) in de-ionized water (5 ml) at room temperature. The mixture was stirred for 15 minutes and the mixture was freeze-dried overnight. The title compound was isolated as a dark red solid material. Yield: 106 mg (79%).

### EXAMPLE 7

### Preparation of Meso-Tetraphenyl Chlorin Disulphonate Bis(Monoethanolamine) ((MEA)₂-TPCS₂ₐ)

Meso-tetraphenyl porphyrin disulphonate bis(triethylamine) prepared from the free acid was dissolved in methanol and an excess of ethanolamine added. The solution was stirred for 15 minutes before the solvent was removed in vacuo at 30° C. with a rotary evaporator. This procedure was repeated two more times.

### EXAMPLE 8

### Preparation of Meso-Tetraphenyl Chlorin Disulphonate Bis(Meglumate) ((Megl)₂-TPCS₂ₐ)

Meso-tetraphenyl chlorin disulphonate (100 mg, 0.13 mmol) was added to a solution of N-methyl-D-glucamine (51 mg, 0.26 mmol) in de-ionized water (5 ml) at room temperature. The mixture was stirred for 15 minutes and the mixture was freeze-dried overnight. The title compound was isolated as a dark red solid material. Yield: 157 mg (100%).

### EXAMPLE 9

### Preparation of Meso-Tetraphenyl Chlorin Disulphonate his (Tris(Hydroxymethyl)Methylamine) ((TRIS)₂-TPCS₂ₐ)

Meso-tetraphenyl chlorin disulphonate (100 mg, 0.13 mmol) was added to a solution of tris(hydroxymethyl)methylamine (31 mg, 0.26 mmol) in de-ionized water (5 ml) at room temperature. The mixture was stirred for 15 minutes and the mixture was freeze-dried overnight. The title compound was isolated as a dark red solid material. Yield: 157 mg (100%).

### EXAMPLE 10

### Solubility of Meso-Tetraphenyl Porphyrin Disulphonate Salts (TPPS₂ₐ)

Water was added in 0.2 ml portions to the various salts described in Examples 1 to 6 (approx. 50 mg) in a test tube. The mixture was agitated until solid particles were broken up and dissolved.

| Example No. | Compound | Minimum solubility in water |
|---|---|---|
| 1 | (MEA)₂-TPPS₂ₐ | 42.3 mg/ml |
| 2 | (Megl)₂-TPPS₂ₐ | 89.7 mg/ml |
| 3 | (TRIS)₂-TPPS₂ₐ | 49.9 mg/ml |
| 4 | (DEA)₂-TPPS₂ₐ | 31.4 mg/ml |
| 5 | (HEP)₂-TPPS₂ₐ | 28.3 mg/ml |
| 6 | (TEA)₂-TPPS₂ₐ | 32.1 mg/ml |

Highly concentrated solutions of TPPS₂ₐ salts were viscous.

### EXAMPLE 11

### Solubility of Meso-Tetraphenyl Chlorin Disulphonate Salts (TPCS₂ₐ)

Water was added in 0.2 ml portions to the various salts described in Examples 7 to 9 (approx. 50 mg) in a test tube. The mixture was agitated until solid particles were broken up and dissolved.

| Example No. | Compound | Minimum solubility in water |
|---|---|---|
| 7 | (MEA)₂-TPCS₂ₐ | 34.9 mg/ml |
| 8 | (Megl)₂-TPCS₂ₐ | 38.9 mg/ml |
| 9 | (TRIS)₂-TPCS₂ₐ | 32.1 mg/ml |

Highly concentrated solutions of TPPS₂ₐ salts were viscous.

### EXAMPLE 12

### Stability of Meso-Tetraphenyl Porphyrin Disulphonate Salts (TPPS₂ₐ)

Aqueous solutions of TPPS₂ₐ salts (approx. 1% weight) were kept at 40° C. for 31 days. The solutions were analyzed by HPLC(HP 1100). The HPLC conditions were as follows:

Column: Agilent Extend C-18

Mobile phase: 85% methanol, 15% water

Flow: 1.0 ml per minute

Detector: UV detector, 415 nm

| Example No. | Compound | Degradation |
|---|---|---|
| 1 | (MEA)₂-TPPS₂ₐ | No degradation |
| 2 | (Megl)₂-TPPS₂ₐ | No degradation |
| 3 | (TRIS)₂-TPPS₂ₐ | No degradation |
| 4 | (DEA)₂-TPPS₂ₐ | No degradation |
| 5 | (HEP)₂-TPPS₂ₐ | No degradation |
| 6 | (TEA)₂-TPPS₂ₐ | No degradation |

Conclusion: all samples were stable at 40° C. for 31 days.

### EXAMPLE 13

### Stability of Meso-Tetraphenyl Chlorin Disulphonate Salts (TPCS₂ₐ)

Aqueous solutions of TPCS₂ₐ salts (approx. 1% weight) were kept at 40° C. for 31 days. The solutions were analyzed by HPLC (HP 1100) according to the method used in Example 12.

| Example No. | Compound | Degradation |
|---|---|---|
| 7 | (MEA)₂-TPCS₂ₐ | No degradation |
| 8 | (Megl)₂-TPCS₂ₐ | No degradation |
| 9 | (TRIS)₂-TPCS₂ₐ | No degradation |

Conclusion: all samples were stable at 40° C. for 31 days.

### EXAMPLE 14

### Capsule Containing (MEA)₂-TPPS₂ₐ for Oral Administration

(MEA)-2-TPPS2a (30 mg) from Example 1 was mixed volumetrically with lactose monohydrate 0.15 mm (900 mg) (Apotekproduksjon AS, Oslo, Norway) using a mortar and pestle. The powder was filled into a hard gelatin capsule no. 000 (Apotekproduksjon AS, Oslo, Norway).

### EXAMPLE 15

### Isotonic Sterile Solution of (TRIS)₂-TPCS₂ₐ without Surfactants for Parenteral or Enteral Administration

(TRIS)-2-TPCS₂ₐ (30 mg) from Example 9 was dissolved in saline (0.9% sodium chloride) (1.0 ml) using a mixer (3M ESP CapMix) for 2 minutes. The brown solution was free from particulates (examined by microscopy).

### EXAMPLE 16

### Kit Comprising (TRIS)₂-TPCS₂ₐ and Solvent for Parenteral or Enteral Administration, e.g. for Administration to the Urinary Bladder or as an Enema

A kit was made comprising two vials:

Composition of vial A: (TRIS)-2-TPCS₂ₐ (20 mg) from Example 9 as dry powder in a vial (100 ml)

Composition of vial B: An aqueous solution (52 ml) comprising:

| | |
|---|---|
| Sodium chloride | 120 mM |
| Potassium dihydrogen phosphate | 4.3 mM |
| Dipotassium hydrogenphosphate | 4.3 mM |
| HCl/NaOH | q.s. ad pH 6.0 |
| Water for injection | q.s |

The solution in vial B was added to vial A, and vial A was shaken by hand for 3 minutes. The solution should be free from visible particles before use.

### EXAMPLE 17

### Topical Formulation Comprising (Tris)₂-TPCS₂ₐ for Administration onto the Skin or Mucosa

(TRIS)-2-TPCS₂ₐ (20 mg) from Example 9 was mixed volumetrically with Unguentum Merck using a mortar and pestle. The brown cream comprising 4 mg (TRIS)-2-TPCS₂ₐ per ml was filled in a glass vial.

### EXAMPLE 18

### Emulsion Formulation Comprising (TRIS)₂-TPCS₂ₐ for Parenteral or Enteral Administration

(TRIS)-2-TPCS₂ₐ (24 mg) from Example 9 was dissolved in a lipid emulsion (ClinOleic 200 mg/ml (20%) from Baxter) using a mixer (3M ESP CapMix) for 2 minutes. The brown emulsion was free from (TRIS)-2-TPCS₂ₐ particulates (examined by microscopy).

### EXAMPLE 19

### Formulation Containing Tetraphenyl Chlorin Disulphonate for PCI Drug Delivery into the Urinary Bladder

TPCS₂ₐ is formulated in aqueous 10% Cremophor ELP to concentrations of 30 or 60 mg/ml according to the following procedure:
TPCS₂ₐ is weighed in a container;
Cremophor ELP is heated to 60-70° C.;
The heated Cremophor is added to the TPCS₂ₐ under stirring conditions;
The solution is stirred for approximately 5 minutes at 60-70° C. and pre-heated (to 60-70° C.) sterile water is slowly added until the Cremophor concentration is 10%.
The solution is kept at 60-70° C. during the whole procedure;
The solution is diluted with Glucose (Fresenius Kabi) (sterile glucose solution with osmolality approx. 290 mOsm/kg); and
The solution is then autoclaved.

The 30 mg/ml formulation may be administered into the urinary bladder using a catheter prior to administration of drug. This is then followed by photoactivation of the photosensitizer using light.

### EXAMPLE 20

### Formulation Containing 30 mg/ml TPCS₂ₐ in 3% Tween 80 for Use as an Enema for PCI Delivery of Drugs to the Lower Part of the Gastrointestinal Tract

TPCS₂ₐ was formulated in 3% Tween 80 according to the following procedure:
TPCS₂ₐ is weighed into a bottle;
50 mM Tris buffer (pH 8.5) is added to the bottle and the solution stirred (500-700 rpm) for 10 minutes;
Tween 80 is added and the solution stirred (500-700 rpm) for 10 minutes. The final concentration of Tween 80 in the formulation is 3%;
Mannitol is added and the solution stirred (500-700 rpm) for 20 hours. The final concentration of mannitol in the formulation is 2.8%;
The formulation is filled into vials with stoppers and caps;
The formulation is then autoclaved for 20 minutes at 121° C.

The formulation should be stored at 2-8° C. protected from light. It may be administered in the form of an enema.

### EXAMPLE 21

### Mucoadhesive Composition Comprising (MEA)₂-TPPS₂ₐ for Direct Use on Body Cavity Walls

(MEA)-2-TPPS₂ₐ (100 mg) from Example 1 was mixed volumetrically with Orabase® paste (5 g) using a mortar and pestle. The brown cream comprising 20 mg (MEA)-2-TPPS₂ₐ per ml was filled in a glass vial. Orabase® paste is a commercial product from Squibb comprising gelatin, pectin, sodium carboxymethyl cellulose, polyethylene and liquid paraffin.

### EXAMPLE 22

### Tablet Composition Comprising (Mea)₂-TPPS₂ₐ for Oral Administration

| | |
|---|---|
| (MEA)₂-TPPS₂ₐ | 100 mg |
| Microcrystalline cellulose | 800 mg |
| Crosscaramellose(Na) (AcDiSol) | 30 mg |
| Magnesium stearate | 30 mg |

All ingredients were blended. A tablet was compressed (tablet diameter: 13 mm; tablet weight: 960 mg).

1-20. (canceled)

21. A method of introducing a drug molecule into the cytosol of a cell located within a body cavity, the method comprising
contacting said cell with a photosensitizing agent and said drug molecule, and
irradiating the cell with light of a wavelength effective to activate the photosensitizing agent.

22. The method of claim 21, wherein said body cavity is a urinary bladder, an oral cavity, a nasal cavity, a female reproductive body cavity system, an abdominal cavity (peritoneum), a lower part of the gastrointestinal system, a cranial cavity, an eye (intravitreous), a lung and bronchial system, or a cavity generated after surgery.

23. The method of claim 22, wherein said body cavity is a urethra, a vaginal cavity, a rectum and/or a colon, or a cavity generated following tumor surgery.

24. The method of claim 21, wherein said photosensitizing agent is selected from a phthalocyanine; a sulphonated tetraphenylporphyrin; nile blue; a chlorin; uroporphyrin I; phylloerythrin; a porpyhrin; methylene blue; a cationic dye; a tetracycline; a naphthalocyanine; a texaphyrine; a pheophorbide; a purpurin; a rhodamine; a fluorescein; a lysosomotropic weak base; and a porphycene.

25. The method of claim 24, wherein said photosensitizing agent is selected from AlPcS₂, AlPcS₂ₐ, TPPS₂ₐ, TPPS₄, TPPS₁, TPPS₂ₒ, a bacteriochlorin, a ketochlorin, a hematoporphyrin, and a benzoporphyrin.

26. The method of claim 24, wherein said photosensitizing agent is selected from TPCS₂ₐ, TPPS₂ₐ, AlPcS₂ₐ, TPPS₄, and porfimer.

27. The method of claim 21, wherein said photosensitizing agent is provided in the form of a pharmaceutically acceptable salt having a water solubility of at least 0.5 mg/ml.

28. The method of claim 27, wherein said salt is formed from a pharmaceutically acceptable base.

29. The method of claim 28, wherein said pharmaceutically acceptable base is an organic amine.

30. The method of claim 29, wherein said organic amine is an amino alcohol.

31. The method of claim 30, wherein said amino alcohol is a lower aliphatic amino alcohol; a cyclic amino alcohol; or an amino sugar.

32. The method of claim 30, wherein said amino alcohol is selected from monoethanolamine, di-ethanolamine, tri-ethanolamine, 2-amino-2-(hydroxymethyl)propane-1,3-diol, 4-(2-hydroxyethyl)-morpholine, 1-(2-hydroxyethyl)-pyrrolidine, glucamine, and N-methylglucamine (meglumine).

33. The method of claim 27, wherein said salt is formed from a pharmaceutically acceptable acid.

34. The method of claim 33, wherein said pharmaceutically acceptable acid is a sulphonic acid.

35. The method of claim 21, wherein said drug molecule is selected from the group consisting of anti-cancer drugs, antibacterial substances, anti-virals, anti-fungal agents, immune-modulating drugs, anti-inflammatories, analgesics, gene therapy agents, oligonucleotides, and gene expression modifying agents.

36. The method of claim 35, wherein said drug molecule is bleomycin.

37. The method of claim 21, wherein the photosensitizing agent and the drug molecule are contacted simultaneously, separately or sequentially.

38. The method of claim 35, wherein said anti-cancer drug comprises a biomolecule prepared by recombinant technology.

39. The method of claim 35, wherein said anti-cancer drug specifically targets gene products over-expressed in target cells, a cellular protein-based target or a cellular non-protein-based target, or which targets an enzyme.

40. The method of claim 35, wherein said immune-modulating drug is selected from protein or peptide antigens for vaccine or immune-stimulating purposes, immune-stimulating oligonucleotides, genes encoding antigens or immune-modulating proteins or peptides, oligonucleotides modifying gene expression in the immune response system, and small molecule drugs having immune-modulating properties.

41. The method of claim 21, wherein said photosensitizing agent and said drug molecule are present in a single pharmaceutical composition.

42. The method of claim 21 which is a method of treating cancer or a method of gene or oligonucleotide therapy.

43. The method of claim 42, wherein the cancer is selected from bladder cancer, ovarian cancer, cervical cancer, lung cancer, brain cancer, colorectal cancer, and cancers of the oral or nasal cavity.

44. The method of claim 21 which is a method of treating infections.

45. The method of claim 44, wherein the infection is caused by a virus, by bacteria, or by fungi.
